(19) United States
(12) Patent Application Publication
(10) Pub. No.: US 2024/0238044 A1
(43) Pub. Date: Jul. 18, 2024

(54) ENDOVASCULAR PLAQUE EXCISION SYSTEM AND EXCISION METHOD

(71) Applicant: Guiyun Zhang, Shanghai (CN)

(72) Inventor: Guiyun Zhang, Shanghai (CN)

(21) Appl. No.: 18/577,475

(22) PCT Filed: Jul. 8, 2022
(86) PCT No.: PCT/CN2022/104692

§ 371 Date: Jan. 8, 2024

(30) Foreign Application Priority Data
Jul. 8, 2021 (CN) 202110773417.1

(51) Int. Cl. A61B 18/24; A61B 1/00; A61B 1/015; A61B 1/018; A61B 1/05; A61B 1/06; A61B 1/313; A61B 17/12; A61B 18/00; A61B 90/00; A61M 25/10

(52) U.S. Cl. CPC A61B 18/245; A61B 1/00154; A61B 1/015; A61B 1/018; A61B 1/05; A61B 1/0684; A61B 1/3137; A61B 17/12045; A61B 2018/00422; A61B 2018/00601; A61B 2018/00982; A61B 2090/376; A61M 2025/1097

(57) ABSTRACT

An endovascular plaque excision system (10), comprising: a balloon catheter system (13) adapted to be inserted into a blood vessel, the balloon catheter system (13) comprising a guide catheter, and a first balloon (131), a second balloon (132) and a third balloon (133) which are disposed at a distal end, the first balloon (131), the second balloon (132) and the third balloon (133) being adapted to be inflated to block the flow of blood in the blood vessel, and the second balloon (132) comprising a blood bypass unit; an endoscope device (12), which comprises an endoscope connecting pipe and an illumination unit and an image acquisition unit which are disposed at a distal end of the connecting pipe, and is adapted to be inserted into the blood vessel via the guide catheter to perform illumination and image acquisition; and an endarterectomy device (14), which comprises an operation unit disposed at a proximal end, an endarterectomy unit disposed at a distal end, and an endarterectomy connecting pipe for connecting the operation unit and the endarterectomy unit, and is adapted to enter the blood vessel via the guide catheter to perform an endarterectomy operation for plaque in the blood vessel.

### FIELD OF THE INVENTION

The present invention relates to an endovascular plaque excision system and an endovascular plaque excision method, and in particular, to an intraarterial plaque excision system and an intraarterial plaque excision method.

### BACKGROUND OF THE INVENTION

The incidence of carotid plaques becomes high. There are data indicating that 200 million patients are suffering from carotid plaques in China, and the detection rate of carotid plaques among people over 60 years old is close to 100%. The carotid plaques, when developed to a certain extent, may lead to carotid artery stenosis or shedding of unstable plaque, which in turn leads to ischemic strokes, seriously threatening the health of people.

At present, the main surgical methods for carotid artery stenosis or occlusion caused by carotid plaques include carotid endarterectomy (CEA) and carotid artery stenting (CAS). The former has advantage in thorough plaque excision, while the latter has advantage in minimally invasive and non-open surgery. However, both surgical methods have some problems.

The main problems unique to CEA itself are as follows: (1) people with poor cardiopulmonary function cannot tolerate such open surgery under general anesthesia; (2) the incidence of cardiac events is relatively high; (3) there are fatal bleeding at the vascular suture. leading to suffocation and cardiac arrest; (4) the incision is likely to be infected; (5) there are defects of carotid sinus nerve injuries and cranial nerve injuries, the serious ones will cause hypertension, hoarseness and disturbance of cervical cutaneous sensation which are difficult to control after surgery; (6) the surgical incision is large, forming scar, and leading to defects of affecting the appearance.

The problems unique to CAS itself are as follows: (1) there is a need for the long-term oral administration of dual anti-platelet aggregation drugs after stent implantation, increasing the risk of bleeding in the nervous system and the digestive system; (2) rather than removing the plaque, a vascular channel is formed by balloon dilatation and squeezing, and a stent is implanted to maintain the shape of the stenotic part and keep it unobstructed, but there still exists the problem of restenosis or reocclusion inside the stent in the long run; (3) the incidence of perioperative plaque shedding and embolic events are relatively high, especially in patients with unstable plaques; (4) for patients who are sensitive to carotid sinus reflex, after stent implantation, it is often the case that heart rate slows down and haemodynamics become unstable for a long time; (5) the stent itself is a metal permanent implant.

Therefore, there is a need for a novel endovascular plaque excision system and corresponding method.

### SUMMARY OF THE INVENTION

The object of the present invention is to provide an endovascular plaque excision system, which may not only achieve the effect of thoroughly excising a plaque in an open visual environment of CEA, completely stripping the intima and a plaque in a lesion region, but also be comparable to the features of CAS minimally invasive surgery, while avoiding the inherent defects of CEA and CAS.

According to an aspect of the present invention, an endovascular plaque excision system is provided, comprising: a balloon catheter system, adapted for being inserted into a blood vessel, comprising a guide catheter, a first balloon, a second balloon, and a third balloon, wherein the first balloon, the second balloon and the third balloon are arranged at a distal end and adapted for being inflated to block blood flow in the blood vessel, and the second balloon comprises a blood shunting unit; an endoscope device, adapted for being inserted into the blood vessel through the guide catheter for illumination and image acquisition, comprising an endoscope connecting tube, an illumination unit, and an image acquisition unit, wherein the illumination unit and the image acquisition unit are arranged at a distal end of the connecting tube; and an intima stripping device, adapted for entering the blood vessel through the guide catheter to perform operation of stripping a plaque in the blood vessel, comprising an operation unit arranged at a proximal end, a stripping unit arranged at a distal end, and a stripping connecting tube which connects the operation unit with the stripping unit.

Preferably, the endovascular plaque excision system further comprises: a laser device, comprising a laser generator and a laser transmission optic fiber, wherein the laser transmission optic fiber is adapted for entering the blood vessel through a optic fiber channel of the endoscope device, and transmitting the laser generated by the laser generator to a selected position for the laser ablation of the plaque in the blood vessel.

Preferably, the endovascular plaque excision system further comprises: a digital subtraction angiography machine, capable of imaging the blood vessel, determining positions where the plaque is generated in the blood vessel, and identifying filling state of the balloons and positions where the balloons are located.

Preferably, the endovascular plaque excision system further comprises: a control device, controlling the balloons to be inflated and the laser generator to generate laser according to preset instructions.

Preferably, the endovascular plaque excision system is adapted for excising a plaque in a carotid artery.

Preferably, the first balloon is adapted for being located at an opening of an external carotid artery of an affected side, the second balloon is adapted for being located in an internal carotid artery of the affected side at distal of the plaque, the third balloon is adapted for being located in the common carotid artery of the affected side at proximal of the plaque, and thereby, after the first balloon, the second balloon and the third balloon are inflated, a blood-free environment is formed in the blood vessel around the plaque.

Preferably, the blood shunting unit of the second balloon is in communication with arterial blood flow to convey the arterial blood flow to distal of the second balloon in an open state.

Preferably, the balloon catheter system comprises a first balloon catheter, a second balloon catheter, and a third balloon catheter.

Preferably, the third balloon catheter is placed into the common carotid artery through a right femoral artery, and the first balloon catheter and the second balloon catheter are placed in parallel alignment into the external carotid artery and the internal carotid artery through a left femoral artery, respectively.

Preferably, the third balloon catheter comprises the guide catheter.

Preferably, the endoscope device is provided with a catheter, which comprises a first lumen for accommodating the illumination unit and the image acquisition unit and a second lumen adapted for the passage of the laser transmission optic fiber and/or irrigation fluid, and a vascular wall protection device is provided at a distal end of the endoscope device.

Preferably, there are a plurality of the image acquisition units, which are evenly and circumferentially distributed and there are a plurality of the illumination units, which are arranged at equal intervals between each two adjacent image acquisition units, respectively.

Preferably, the endoscope device further comprises a third channel served as a working channel.

According to another aspect of the present invention, an endovascular plaque excision system is provided, which comprises: a balloon catheter system, adapted for being inserted into a blood vessel, comprising a guide catheter, a first balloon, a second balloon, and a third balloon, wherein the first balloon, the second balloon and the third balloon are arranged at a distal end, and are adapted for being inflated to block blood flow in the blood vessel, and the second balloon comprises a blood shunting unit; an endoscope device, adapted for being inserted into the blood vessel through an artery sheath for illumination and image acquisition, comprising an endoscope connecting tube, an illumination unit, and an image acquisition unit, wherein the illumination unit and the image acquisition unit are arranged at a distal end of the connecting tube; and an intima stripping device, adapted for entering the blood vessel through a working channel of the endoscope device to perform operation of stripping a plaque in the blood vessel, comprising an operation unit arranged at a proximal end, a stripping unit arranged at a distal end, and a stripping connecting tube which connects the operation unit with the stripping unit.

Preferably, the endovascular plaque excision system further comprises: a laser device, comprising a laser generator and a laser transmission optic fiber, wherein the laser transmission optic fiber is adapted for entering the blood vessel through the working channel of the endoscope device, and transmitting the laser generated by the laser generator to a selected position for laser ablation of the plaque in the blood vessel.

Preferably, the endovascular plaque excision system further comprises: a digital subtraction angiography machine, capable of imaging the blood vessel, determining the position where the plaque is generated in the blood vessel, and identifying the filling state of the balloons and the positions where the balloons are located.

Preferably, the endovascular plaque excision system further comprises: a control device, controlling the balloons to be inflated and the laser generator to generate laser according to preset instructions.

Preferably, the endovascular plaque excision system is adapted for excising a plaque in a carotid artery.

Preferably, the first balloon is adapted for being located at an opening of an external carotid artery of an affected side, the second balloon is adapted for being located in an internal carotid artery of the affected side at distal of the plaque, the third balloon is adapted for being located in a common carotid artery of the affected side at proximal of the plaque, and thereby, after the first balloon, the second balloon and the third balloon are inflated, a blood-free environment is formed in the blood vessel around the plaque; and a carotid sheath is arranged in the common carotid artery at distal of a target blocked by the third balloon, so that the endoscope device is able to enter the common carotid artery and the internal carotid artery.

Preferably, the blood shunting unit of the second balloon is in communication with arterial blood flow to convey the arterial blood flow to distal of the second balloon in an open state.

Preferably, the balloon catheter system comprises a first balloon catheter, a second balloon catheter, and a third balloon catheter.

Preferably, the third balloon catheter enters the right common carotid artery through a right femoral artery, and the first balloon catheter and the second balloon catheter enter the third balloon catheter in parallel alignment, and then enter the external carotid artery and the internal carotid artery respectively after exiting the third balloon catheter.

Preferably, the third balloon catheter comprises the guide catheter.

Preferably, the endoscope device has a double-lumen design, comprising an inner lumen and an outer lumen which are coaxially arranged, wherein the inner lumen may optionally be a working channel, through which a corresponding instrument, such as a plaque stripping instrument, a breaking instrument, or a grasping instrument, may be fed and the surgical field irrigation may be performed, and the outer lumen is adapted for serving as a layout channel for an endoscopic illumination system and a camera system.

Preferably, the endoscope device is provided with a plurality of the image acquisition units which are evenly and circumferentially distributed along the outer lumen of the endoscope device, and a plurality of the illumination units which are arranged at equal intervals between each two adjacent image acquisition units, respectively.

According to another aspect of the present invention, a method for operating the aforementioned endovascular plaque excision system is provided, comprising: inserting the first balloon, the second balloon and the third balloon of the endovascular plaque excision system into the carotid artery, so that the first balloon is located at the opening of the external carotid artery of the affected side, the second balloon is located in the internal carotid artery of the affected side at distal of the plaque, and the third balloon is located in the common carotid artery of the affected side at proximal of the plaque; sequentially filling the second balloon, the first balloon, and the third balloon; and once blood flows back into the second balloon catheter, opening the blood shunting unit arranged in the second balloon.

Preferably, the method further comprises: operating the intima stripping device after the first balloon, the second balloon and the third balloon are inflated.

Preferably, the method further comprises: operating the laser device to guide laser to the plaque in the carotid artery.

Preferably, the method further comprises: guiding irrigation fluid into the carotid artery.

According to another aspect of the present invention, a method for excising a plaque in the common carotid artery is provided, comprising: puncturing left and right femoral arteries via transfemoral approach, and placing artery sheaths respectively; placing the third balloon catheter into the common carotid artery of an affected side through the right femoral artery sheath, then placing the first balloon catheter into an external carotid artery of the affected side through the left femoral artery sheath, and then placing the second balloon catheter into an internal carotid artery of the affected side through the left femoral artery sheath, so that the first balloon is located at an opening of the external carotid artery of the affected side, the second balloon is located in the internal carotid artery of the affected side at distal of the plaque, and the third balloon is located in the common carotid artery of the affected side at proximal of the plaque; sequentially filling the second balloon, the first balloon, and the third balloon, opening a flank of a Y-type valve at a tail end of the second balloon catheter, and connecting the second balloon catheter with a communication tube of the left femoral artery sheath once blood is seen to flow back, so that arterial blood in the left femoral artery sheath flows into the internal carotid artery of the affected side through the second balloon catheter; introducing an endoscope device into the common carotid artery through the third balloon catheter or introducing an endoscope device into the common carotid artery through a carotid sheath to reach the proximal of the plaque; placing an intima stripping device through a working lumen of the endoscope device and mechanically stripping the proximal of the plaque to separate it from media, laser ablating the plaque tissue, and performing irrigation to make debris flow out of the working lumen of the endoscope device.

Preferably, the method for excising a plaque in the artery further comprises: continuously stripping the plaque distally, followed by laser ablation and irrigation until the plaque is completely excised under the endoscopic vision, and performing thorough irrigation to ensure that the surgical field is clean and free of debris.

Preferably, the method for excising a plaque in the artery further comprises: after the excision is completed, keeping a tail end of the third balloon catheter open, first emptying the second balloon and then immediately filling it again, then emptying and withdrawing the first balloon, appropriately sucking the third balloon catheter, then emptying the third balloon, and finally emptying and withdrawing the second balloon.

Preferably, the method for excising a plaque in the artery further comprises: performing angiography again through the third balloon catheter to confirm that the internal carotid artery and the intracranial artery are unobstructed.

The endovascular plaque excision system is mainly used in the fields of coronary arteries and peripheral blood vessels. However, in both fields, the endovascular plaque excision system is used under X-ray fluoroscopy, and the surgical area cannot be viewed directly, and thus it is prone to cause vascular penetration. Moreover, the endovascular plaque excision system can only partially ablate the plaque raised into the vascular lumen, but cannot completely ablate the thickened intima and plaque, and therefore the plaque will still grow, resulting in postoperative restenosis or reocclusion, and also the vascular wall can be easily injured under non-direct vision.

The endovascular plaque excision system according to an embodiment of the present invention realizes surgery under the visual environment of a vascular endoscope, achieves the same immediate surgical result as CEA, and ensures that the vascular wall is prevented from being injured by laser. The endovascular plaque excision system according to the present invention, which belongs to a minimally invasive interventional surgery system, does not require to incise the skin of the neck and separate tissues, takes the advantages of both CEA and CAS, but avoids the disadvantages of CEA and CAS successfully, dispenses with a permanent implant in the body, and reduces or eliminates the need for long-term dual anti-platelet aggregation therapy. In addition, it is convenient to implement a remedial technique, e.g. it is possible to release a carotid artery stent or a covered stent through a balloon catheter.

The excision of a carotid plaque employing the endovascular plaque excision system according to the present invention mainly comprises: via the transfemoral approach, using an improved flexible electronic endoscope as the illumination and video acquisition system in the carotid artery lumen, orientationally removing the plaque raised into the lumen by means of an optic fiber laser transmission system under a visual environment, physically stripping the intima in the lesion region, and further removing the separated intima tissue by laser with the help of a vascular wall protection device. Thus, the intima and the plaque are thoroughly removed, achieving the same effect as CEA, and it is ensured that the vascular wall is protected from being injured by laser.

The novel endovascular plaque excision system according to the present invention has unique innovativeness:

(1) Endoscope system: According to various embodiments of the present invention, the endoscope device can be provided in two ways. The first endoscope device is adapted for the transfemoral approach. It is the first time that a high-resolution (4K) flexible electronic endoscope is applied to the endoluminal ablation of carotid plaques. The endoscope features that it has a head end turnable to the left or right by up to 90 degrees in the same plane. By rotating the endoscope catheter and changing the orientation of the head end of the endoscope, it can achieve the full-angle observation of the carotid wall and the plaque, providing fine and ultra-high definition videos for the operation of intimal plaque stripping. The real-time video effect achieved by the endoscope is comparable to an open surgery under a microscope (CEA). The greatest advantage is that a full-angle vision can be obtained in the arterial lumen without incising the carotid artery wall in an open way. The greatest innovation of the endoscope is that the endoscope device (6F or 12F) is provided with a vascular wall protection device at the distal end, which can protect the vascular wall to the greatest extent from being accidentally injured by laser and improve work efficiency. The second endoscope device, which is adapted for the transcervical approach, has a double-lumen structure which has coaxial inner and outer lumens, and the inter lumen as working lumen has larger diameter, facilitating the entry and operation of an instrument. The multi-point illumination and camera system is arranged in the outer lumen, so that the surgical field observation is clearer and broader.

(2) Balloon blocking and shunting unit: In order to acquire clear images, the vascular endoscope needs to be in a blood-free aqueous environment. Forward blood flow in the common carotid artery and backflow in the external carotid artery, the superior thyroid artery and the internal carotid artery are blocked by the three balloons in separated way, so a blood-free environment centered on the plaque can be achieved by the irrigation system of the endoscope. In the aqueous environment, a surgeon can perform accurate laser ablation of the plaque by means of observation under the ultra-high definition endoscope. Because of the introduction of the second balloon catheter, it can block the blood backflow in the internal carotid artery and achieve a blood-free environment in the blood vessel of the lesion region along with the first and the third balloon catheter, and more importantly, it can provide the blood shunting function (i.e. the shunting unit) at the affected side, which ensures the supply of blood to the brain at the distal end of the affected side and thus relatively sufficient and unlimited operation time, and therefore is different from the shunt commonly used in CEA. Both ends of the shunt commonly used in CEA comprise a balloon blocking the proximal end of the common carotid artery and a balloon blocking the proximal end of the internal carotid artery, respectively, both of which are placed by incising the common carotid artery and the internal carotid artery and are not applicable for the system according to the present invention.

(3) full-vision visibility+anatomical separation+laser ablation: The endoluminal plaque excision effect achieved by the system according to the present invention is comparable to conventional CEA. The conventional endoluminal laser ablation for the coronary arteries of the heart is to partially ablate a plaque by pushing a laser ablation catheter along a micro-guide wire under DSA (under non-direct view), with most of the plaque removed only, and it cannot make the whole plaque separate from the media, and thus cannot achieve the removal of the plaque at the anatomical level. Since the diameter of the peripheral blood vessel is usually less than that of the carotid artery, it is common to achieve proximal blockage with only a single balloon. Because of distal collateral anastomosis, it is difficult to ensure that there is absolutely no blood at the plaque, which affects endoscopic observation. Therefore, at present, the clinical laser ablation of arterial plaques in the lower limbs is still similar to the laser ablation of plaques in the coronary arteries of the heart, and it is difficult to achieve complete visibility under the endoscope, so it is impossible to excise plaques at the anatomical level. In contrast, when the system according to the present invention is used to excise a carotid plaque, under the cooperation of DSA+endoscope+three-balloon blocking+shunting+mechanical stripping+vascular wall protection+laser ablation, the endoluminal excision of the carotid plaque can be fully realized at the anatomical level in a visible environment.

(4) In case of transcervical approach, the first balloon catheter and the second balloon catheter are preferably introduced in parallel alignment into the third balloon catheter and delivered to their respective blocking targets, respectively. Once the third balloon catheter is placed in place, it is convenient for the first and second balloon catheters to be placed in place. Further, catheter occupation between the third balloon and the vascular wall in case of transfemoral approach, caused by the parallel alignment, is eliminated, so that the third balloon can be better directly attached to the vascular wall and the blocking is more accurately.

(5) In case of the transcervical approach, the diameter of the working channel of the endoscope device is increased to a greater extent, and the effective length of the endoscope entering the blood vessel is greatly reduced, facilitating the corresponding surgical instrument getting in and out and improving the working efficiency. The problems which the endoscope system would encounter in case of the transfemoral approach, such as long path, tortuous blood vessels and the difficulty in passing through, are avoided.

The operation steps of using the system according to the present invention for carotid plaque excision are as follows (taking the case of transfemoral approach as an example): making a patient lie flat on a DSA bed, with general anesthesia, and routinely disinfecting and putting a towel; taking puncture points at the left and right femoral arteries, incising the skin, puncturing the left and right femoral arteries by means of an improved Seidinger technique, and placing 15F artery sheaths, respectively; under DSA fluoroscopy, placing the third balloon (with high compliance) catheter into the common carotid artery of the affected side through the right femoral artery sheath (15F) under the guidance of a guide wire, and placing the first balloon (with high compliance) catheter into the external carotid artery of the affected side through the left femoral artery sheath (15F) under the guidance of a guide wire, and then placing the second balloon (with high compliance) catheter into the internal carotid artery of the affected side through the left femoral artery sheath (15F) under the guidance of the guide wire. Filling the second balloon, the first balloon and the third balloon in sequentially, and injecting contrast medium through the third balloon catheter to confirm that the blocking by each balloon is accurate. Finally, opening the flank of the Y-type valve at the tail end of the second balloon catheter, and connecting the second balloon catheter to the communication tube of the left femoral artery sheath once blood is seen to flow back, so that the arterial blood in the left femoral artery sheath flows into the internal carotid artery of the affected side through the second balloon catheter. Under DSA fluoroscopy, introducing the endoscope system slowly into the common carotid artery through the third balloon catheter to reach the proximal of the plaque. Irrigating the surgical field centered on the plaque through the endoscope, and confirming that the blocking by each balloon is accurate again under the endoscope. Afterwards, placing the intima stripping device through the working lumen of the 12F endoscope and mechanically stripping the proximal of the plaque to separate it from the media, and laser ablating the plaque tissue in the vascular wall protection device at the head end of the endoscope, and performing continuous irrigation, so that debris flow out from the working lumen of the 12F endoscope. Continuously stripping the plaque distally, followed by laser ablating and irrigating until the plaque is completely excised under the endoscope, and thoroughly irrigating to ensure that the surgical field is clean and free of debris. After the surgery is completed, keeping the tail end of the third balloon catheter open, first emptying the second balloon and then immediately filling it again, then emptying and withdrawing the first balloon, appropriately sucking the third balloon catheter, emptying the third balloon, and finally, emptying and withdrawing the second balloon. Before completing the surgery, performing angiography again through the third balloon catheter to confirm that the internal carotid artery and the intracranial artery are unobstructed.

### BRIEF DESCRIPTION OF THE DRAWINGS

The present invention will be more fully understood from the following detailed description in combination with the accompanying drawings, in which like elements are numbered in a similar manner and among which:

FIG. 1 is a schematic structural diagram of an endovascular plaque excision system according to an embodiment of the present invention;

FIG. 2 is a schematic diagram of an endovascular plaque excision system operating in a human body according to an embodiment of the present invention;

FIG. 3 is an enlarged view of A in FIG. 2;

FIG. 4 is a diagram of the experimental therapeutic effect of the endovascular plaque excision system according to an embodiment of the present invention;

FIG. 5 is a schematic diagram of a transcatheter microscopic stripper used in the endovascular plaque excision system according to an embodiment of the present invention;

FIGS. 6, 7 and 8 are schematic diagrams of a vascular wall protection device used in the endovascular plaque excision system according to an embodiment of the present invention; and

FIGS. 9, 10 and 11 are schematic structural diagrams of an endoscope device according to an embodiment of the present invention.

### DETAILED DESCRIPTION OF THE INVENTION

The technical solution of the present invention will be further illustrated in detail below through embodiments in combination with the accompanying drawings, while the present invention is not limited to the following embodiments.

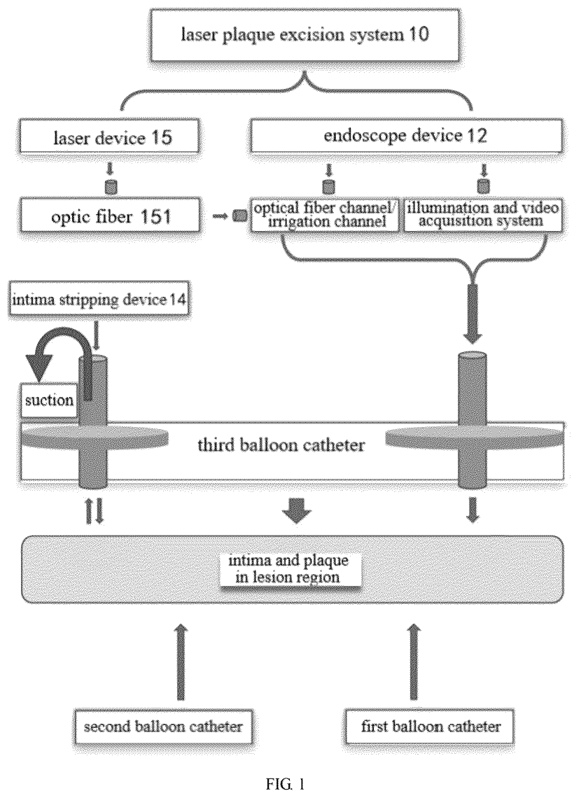

FIG. 1 is a schematic structural diagram of an endovascular plaque excision system according to an embodiment of the present invention, which is used for the laser excision of a plaque in a blood vessel via transfemoral approach.

As shown in FIG. 1, the present invention discloses an endovascular plaque excision system 10, which may include an endoscope device 12, a balloon catheter system 13, and an intima stripping device 14, and may also include a laser device 15 for laser generation and transmission.

The balloon catheter system 13 may be placed into a blood vessel of the human body through sheaths 16, and may be used for blood flow blocking (131), for blood flow blocking and shunting (132), or for blood flow blocking and functioning as a guide catheter (133). According to an embodiment of the present invention, the balloon catheter system 13 may include three balloons, e.g. a guide catheter and a first balloon 131, a second balloon 132 and a third balloon 133 arranged at the distal end. As described below, the first balloon, the second balloon and the third balloon are adapted for being inflated to block blood flow in the blood vessel, and the second balloon further includes a blood shunting unit. The first balloon 131, the second balloon 132 and the third balloon 133 may have respective guide catheters which form a first balloon catheter, a second balloon catheter, and a third balloon catheter, respectively.

During operation, for example, the balloon catheter system 13 may be used for guiding the endoscope device 12, the intima stripping device 14 and the laser device 15 into the body. In an embodiment, the endoscope device 12 enters the human body through the third balloon catheter.

The endoscope device 12 may be an improved flexible electronic endoscope, which may illuminate and acquire a video in a blood vessel, such as a carotid artery lumen. Through the blood flow blocking by the balloons 131, 132 and 133 and the introduction of the endoscope device, it is able to achieve endovascular surgery under a blood-free and visual environment is realized, reaching the same immediate surgical result as CEA.

According to a preferred embodiment of the present invention, the second balloon catheter may be a double-lumen catheter; one of lumens communicates with the balloon, and the contrast medium may be filled through the lumen so as to inflate the balloon; and the other lumen is a main channel in which an instrument, such as a microcatheter or a micro-guide wire, may be inserted or fluid may be injected or arterial blood from the femoral artery may be shunted, to ensure forward blood flow in the distal vascular bed at the position blocked by the balloon. Because of the introduction of the second balloon catheter, it can block blood backflow in the internal carotid artery and achieve a blood-free environment in the blood vessel of the lesion region along with the first and the third balloon catheter, and more importantly, it can provide a blood shunting function at the affected side, which ensures the supply of blood to the brain at the distal end of the affected side and thus relatively sufficient and unlimited operation time.

The intima stripping device 14 may be, for example, a microscopic instrument for surgically stripping intima under the endoscope, such as a transcatheter microscopic stripper. Referring to FIG. 5, the transcatheter microscopic stripper is integrally formed from a silver-titanium alloy wire, which may be divided into two portions: a distal end and a proximal end. The distal end is a stripping shovel 41, a shovel surface 42 of which is made by cutting a silver-titanium alloy wire with a diameter of 1 mm to 1.5 mm at an angle of 15 to 45 degrees, such as 15 degrees, 30 degrees, or 45 degrees, etc. with the sharp edges around the cutting face ground to be blunt. As required, the stripping shovel 41 and an operating rod 43, which is the proximal end, are bent into an angle of 5 to 45 degrees, such as an angle of 5 degrees, 15 degrees, 30 degrees, or 45 degrees, along the direction of the shovel surface 42, and the distance from the shovel end to the bending point may be varied, such as 5 mm, 10 mm, 15 mm, or 20 mm. Alternatively, the intima stripping device 14 may be a transcatheter microscopic tissue capture device, which consists of a storage net, a capture ring, and a feed rod. The storage net is made of nylon or other membranous materials with a mesh diameter of 50 μm to 100 μm, while the capture ring and the feed rod are made of silver-titanium alloy, and form an angle of 135 degrees therebetween. The intima stripping device 14 may also be other suitable devices capable of stripping the intima and the plaque.

The intima stripping device 14 may strip the intima and the plaque 17 in the lesion region, and completely strip the plaque 17 in the lesion region. Plaque stripping or intima stripping according to the present invention refers to stripping from an anatomical structure, i.e. stripping from a potential gap between the intima and the media at the plaque, which is implemented in a physical method and thus cause no thermal injury to the vascular wall.

According to a preferred embodiment of the present invention, the laser device 15 includes a laser generator and a laser transmission optic fiber 151. The laser generator is used for emitting laser and controlling the emitted laser. The laser transmission optic fiber 151 guides the laser to the stripped plaque region, so as to ablate the plaque with the laser. The laser optic fiber 151 reaches the stripped plaque region through an optic fiber chamber (also functioning as an irrigation channel) of the endoscope device 12.

According to an embodiment of the present invention, the endoscope device 12 is provided with a catheter, which includes at least two chambers, and a laser transmission optic fiber is arranged in one of the chambers, which may also be used for the passing of irrigation fluid. In this way, on one hand, the irrigation fluid may irrigate the ablated plaque debris, and on the other hand, may decrease the temperature in the laser transmission optic fiber 151 and the blood vessel in the working region so as to reduce thermal injury to the vascular wall. A light source for illumination and a video acquisition device are arranged in the other chamber of the endoscope device 12.

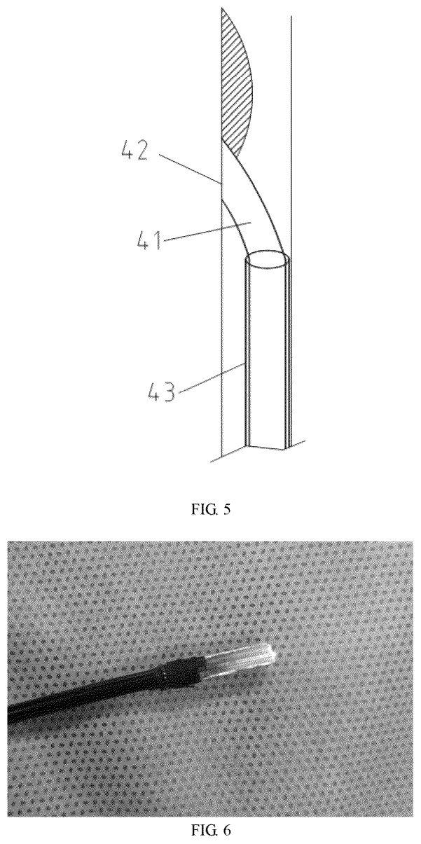

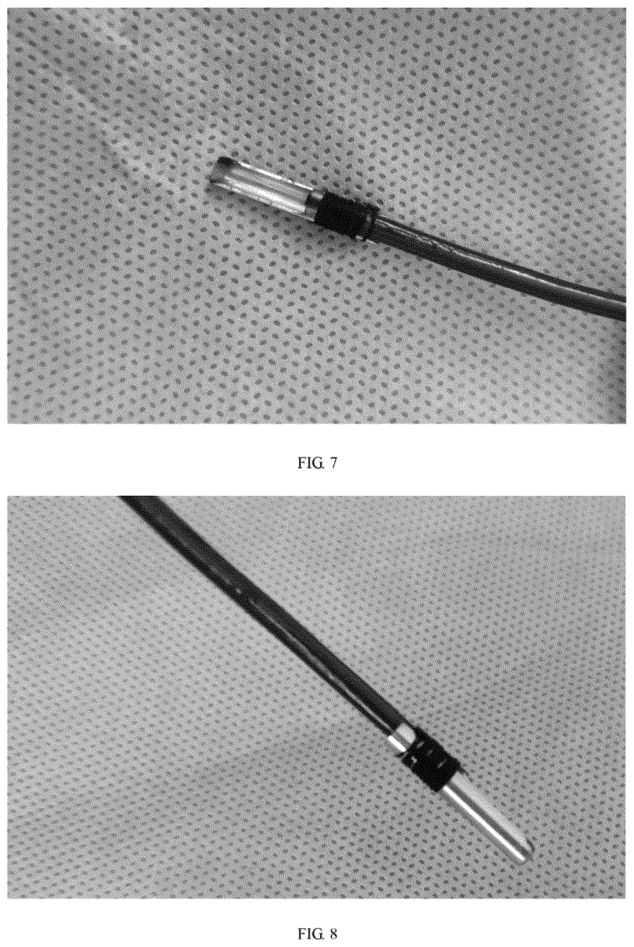

Preferably, the endoscope device 12 is a double-lumen system with a diameter of 6F (or a three-lumen system with a diameter of 12F, which has one more working channel than the double-lumen system with a diameter of 6F, with such working channel, the stripping device may be fed through, and also the plaque debris may be sucked or taken out via the capture device), with one lumen for an endoscopic illumination system and a camera system and the other lumen as a channel for optic fiber entry and irrigation. The head end has a flexible structure of 2 cm, which may be operated in vitro to change the orientation of the lens. The distal end of the endoscope device 12 may further be provided with a vascular wall protection device. For example, as shown in FIGS. 6 to 8, the vascular wall protection device is a thin-walled stainless steel tube embedded at the periphery of the head section of the endoscope along ½ to ⅔ of the circumference. During operation, the device may isolate the separated plaque from the vascular wall, provide the best protection for the laser transmitted by the optic fiber to act on the target, and prevent the laser from causing thermal injury to the vascular wall. The head end of the optic fiber is located within the protection device, with a distance of 1 mm from the head end of the protection device. The distal end of the laser transmission optic fiber 151 is located within the vascular wall protection device, so that the laser transmission optic fiber 151 will cause no injury to the vascular wall during ablation. F is the size unit related to catheter (the abbreviation of French), and 1F is about 0.33 mm.

According to an embodiment of the present invention, the endovascular plaque excision system 10 may further include a digital subtraction angiography machine (DSA), which may image the blood vessel, determine the position of the lesion region (plaque) and the diameter of the blood vessel, and select from balloon catheters of various specifications according to the diameter. The third balloon catheter, the second balloon catheter and the first balloon catheter are introduced through guide wires under DSA fluoroscopy. After the balloon catheters are introduced in place, the filling extent of the balloons are monitored under fluoroscopy, and whether the balloons completely block blood flow is determined by means of contrast. The endoscope device 12 is then introduced through the third balloon catheter.

According to an embodiment of the present invention, the balloon catheter system 13 may include three balloon catheters, i.e. a first balloon catheter (2F), a second balloon catheter (4F), and a third balloon catheter (10F or 12F). The balloon catheter system 13 may also be an integrated balloon catheter system. The so-called integrated balloon catheter system means that the catheter portions of the first and the second balloon catheter are embedded in the catheter wall of the third balloon catheter via a certain process or completely seamlessly wrapped in the balloon of the third balloon catheter, with the advantage that the third balloon has a more accurate blocking effect on blood flow in the common carotid artery.

As shown in FIGS. 1, 2 and 3, the front end of the balloon catheter is provided with an inflatable balloon, which may be a high-compliance balloon. After the three balloon catheters are introduced into the blood vessel of the human body through the sheaths 16, the first balloon 131 is placed at the opening of the external carotid artery of the affected side, the second balloon 132 is placed in the narrow portion of the internal carotid artery which is distal, and the third balloon 133 is placed in the common carotid artery, i.e. the proximal of the plaque. The balloon 131 at the opening of the external carotid artery may block blood backflow in the external carotid artery and the superior thyroid artery, the balloon 132 in the narrow portion of the internal carotid artery which is distal may block blood backflow in the internal carotid artery, and the balloon 133 in the common carotid artery can block blood flow in the common carotid artery. By filling the three balloons 131, 132 and 133, a blood-free environment is achieved in the vascular lumen in the lesion region. The catheter of the balloon 133 in the common carotid artery may be used as a guide catheter for guiding the endoscope device 12, the intima stripping device 14 and the laser device 15 into the lesion region in the body, or may be used as a suction catheter for sucking debris.

According to an embodiment of the present invention, the endovascular plaque excision system 10 may further include a continuous irrigation and suction device. In terms of irrigation, irrigation fluid may be delivered into the body through one of the chambers of the endoscope device 12 (6F endoscope), and in terms of suction, the debris in the body may be sucked through the third balloon catheter (or through the working channel of the 12F endoscope).

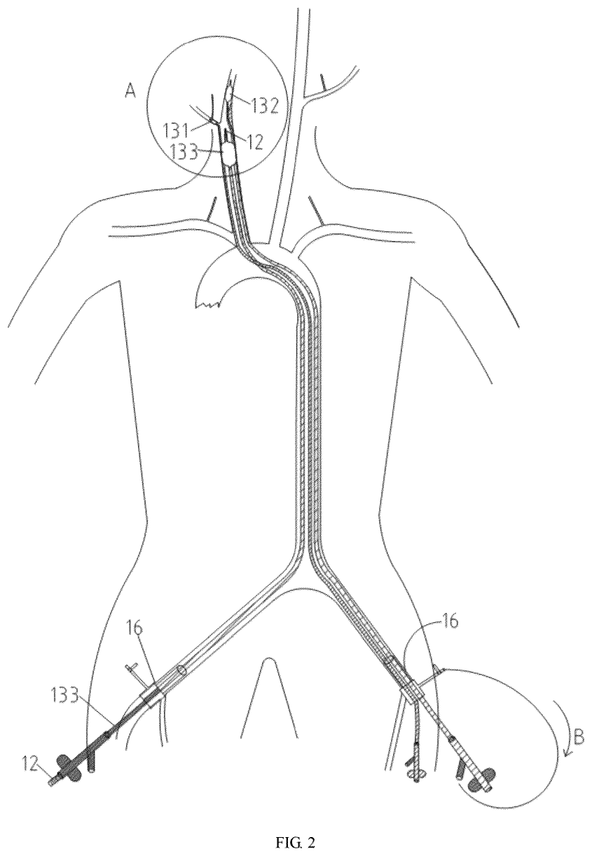

FIG. 2 is a schematic diagram of the endovascular plaque excision system operating in the human body according to an embodiment of the present invention. As shown in FIG. 2, when using the endovascular plaque excision system 10 according to one embodiment of the present invention, firstly, with the help of the digital subtraction angiography machine, the artery sheaths 16 are placed in place respectively via the left and right femoral artery puncture, and the first and the second balloon catheters are introduced through the left femoral artery sheath under the condition of continuous pressurized irrigation, and the first and the second balloons are located at the opening of the external carotid artery of the affected side and in the narrow portion of the internal carotid artery which is distal, respectively. The third balloon catheter is introduced through the right femoral artery sheath 16, and the third balloon is located in the common carotid artery of the affected side, i.e., at the proximal of the plaque. Preferably, before filling, the side tube of the left artery sheath is connected with the flank of the Y-type valve at the tail end of the balloon catheter 132, and the three-way cock is opened, so that the blood in the left femoral artery flows into the internal carotid artery which is at the distal of the affected side through the balloon catheter 132. The left and right femoral artery sheaths are both 15F artery sheaths which are 20 cm long, with main channels allowing catheters or guide wires within 15F to enter and side tubes provided with three-way cocks. After the three-way cocks are opened, the blood flow in the femoral arteries may flow out from the side tubes, and through the connection with the flank of the Y-shaped valve at the tail end of the second balloon catheter, the blood flow in the left femoral artery may be partially guided to the second balloon catheter, so that blood backflow in the internal carotid artery can be blocked by filling the balloon 132 of the head end of the second balloon catheter and forward blood flow in the internal carotid artery is ensured as well, providing effective perfusion for the brain of the surgical side. Therefore, the surgical operation can be carried out leisurely, almost without time limitation. The balloon 132 is first inflated to block blood flow in the internal carotid artery, so as to ensure the effective perfusion of the cerebral hemisphere of the affected side. The balloon 131 in the external carotid artery is then inflated to block blood backflow in the external carotid artery and the superior thyroid artery, and finally, the balloon 133 located in the common carotid artery of the affected side is inflated, thereby achieving a blood-free environment in the vascular lumen in the lesion region.

Then, the intima stripping device 14 is fed into the lesion region through the third balloon catheter to strip the intima and the plaque in the lesion region. After stripping, the laser generator is turned on and through the laser transmission optic fiber 151, the emitted laser ablates the stripped intima and plaque. For a plaque with a large volume, it is also possible to perform endoluminal laser ablation, followed by mechanical stripping, and then ablate the stripped tissue, and the large tissue debris produced may be taken out by the transcatheter microscopic tissue capture device until the intima and the plaque in the lesion region are thoroughly removed. The carotid endarterectomy is completed under continuous irrigation by the endoscope device 12 and suction by the third balloon catheter, the carotid endarterectomy is completed, thoroughly excising the thickened intima and plaque tissue in the lesion region, and thereby achieving the purpose of removing the lesion at the anatomical level.

Components related to the blood shunting function of the second balloon catheter may be collectively referred to as a blood shunting unit. For example, the blood shunting unit may include the main channel of the second balloon catheter or the catheter in the main channel, a suitable control valve (e.g. the aforementioned three-way valve or Y-type valve, etc.), etc. The main channel or the catheter in the main channel introduces blood at the femoral artery and leads out the blood beyond the distal end of the second balloon. A device, such as the control valve, is used to control the shunting of blood. The direction shown by B in FIG. 2 is the blood shunting direction according to one embodiment of the present invention.

According to the present invention, since the blood flow can return to the brain by means of shunting, the carotid plaque stripping surgery can be implemented under the condition that the blood vessel in the lesion region is blocked without stopping the blood flow in the brain.

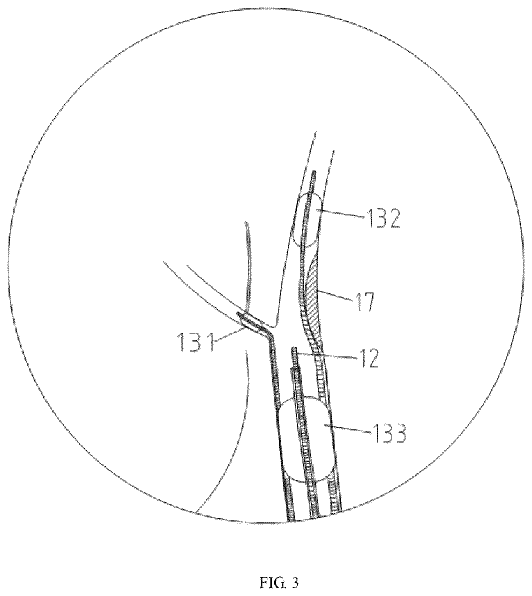

FIG. 3 is a diagram of the in-vitro experimental therapeutic effect employing the endovascular plaque excision system according to an embodiment of the present invention. As shown in FIG. 3, with the endovascular plaque excision system 10 according to the present invention, it is possible to achieve the same surgical result as CEA, i.e. the complete excision of the intima and the plaque, under minimally invasive conditions comparable to conventional percutaneous carotid artery stenting. Under the protection of the vascular wall protection device at the head end of the endoscope, the plaque can be thoroughly ablated by laser without injuring the vascular wall. FIG. 3 shows the intact state of the vascular wall after the thorough excision of the plaque tissue by laser ablation performed under the endoscope provided with the vascular wall protection device in the state of simulated stenosis or occlusion due to the carotid plaque in vitro (the model is made of porcine aortic vessel embedded with human carotid plaque obtained surgically). This experiment proves that the laser excision of a carotid plaque is safe and feasible with the help of the vascular wall protection device.

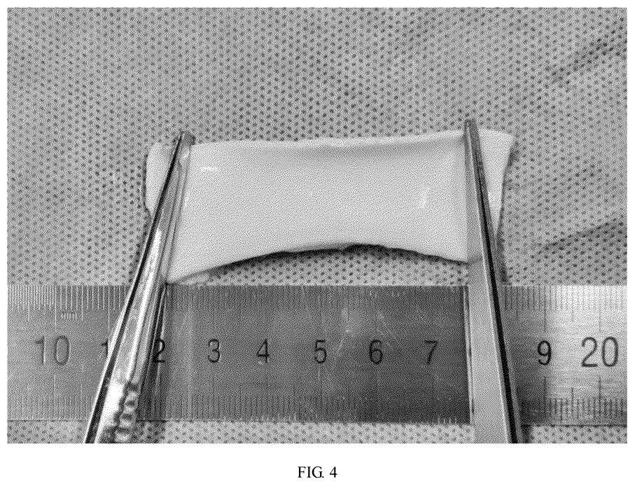

FIG. 4 is a diagram of the experimental therapeutic effect of the endovascular plaque excision system according to an embodiment of the present invention.

According to another embodiment of the present invention (i.e. in case of the transcervical approach), the endoscope device 12 enters the common carotid artery and the internal carotid artery through the artery sheath located in the common carotid artery, and the endoscope handle is held by hand or there provides a corresponding device for fixing the endoscope in vitro, facilitating keeping a good surgical field. The puncture point where the artery sheath enters the common carotid artery is at or below the level of thyroid cartilage, and it is ensured that such puncture point is located at the distal of the target blocked by the third balloon, and the inner diameter of the artery sheath should match the outer diameter of the endoscope.

The designed maximum length of the portion of the endoscope device 12 entering the blood vessel is 15 cm, and generally, the effective length entering the blood vessel is 5 cm. The 2 cm portion of the head end is of a flexible structure, the maximum deflection angle of which is 45 to 60 degrees. The endoscope device 12 may include an inner lumen and an outer lumen which are arranged coaxially. Optionally, the designed maximum outer diameter of the endoscope device 12 may be 12F, 14F, 16F, or 18F, and the inner diameter of the respective inner lumen may be 5F, 7F, 9F, or 11F. The diameter of the working channel of the endoscope is increased to a greater extent, and the effective length of the endoscope entering the blood vessel is greatly reduced, facilitating a corresponding surgical instrument getting in and out, and improving the working efficiency. The problems which the original endoscope system would encounter in case of the transfemoral approach, such as long path, tortuous blood vessels and difficulty in passing through, are avoided.

The inner lumen may optionally be a working channel through which a corresponding instrument, such as a plaque stripping instrument, a breaking instrument, or a grasping instrument, may be fed and the surgical field irrigation may be performed. The outer lumen may optionally be a layout channel for the endoscopic illumination system and the camera system. The endoscopic illumination system includes a plurality of illumination units, and the camera system includes a plurality of image acquisition units. The plurality of image acquisition units are evenly and circumferentially distributed along the outer lumen of the electronic endoscope, and the plurality of illumination units are arranged at equal intervals between each two adjacent image acquisition units, respectively.

In this way, the diameter of the working channel of the endoscope is increased to a greater extent, and the effective length of the endoscope entering the blood vessel is greatly reduced, facilitating a corresponding surgical instrument getting in and out, and improving the working efficiency. The problems which the endoscope system would encounter in case of the transfemoral approach, such as long path, tortuous blood vessels and difficulty in passing through, are avoided.

Preferably, the outer lumen is divided into 12 equal parts, and the plurality of image acquisition units are located at 3 o'clock, 6 o'clock, 9 o'clock and 12 o'clock, respectively. Acquired images may be synthesized into a holographic real-time image by a computer software system, or may be displayed on four external monitors respectively, facilitating observation by the surgeon. The plurality of illumination units are located at 1 o'clock, 2 o'clock, 4 o'clock, 5 o'clock. 7 o'clock, 8 o'clock, 10 o'clock and 11 o'clock, respectively. Such layout of the outer lumen may ensure good surgical field during operation in the vascular lumen, avoiding the possibility that a single camera system cannot see the surgical field clearly because of a special angle and the obstruction of corresponding tissues.

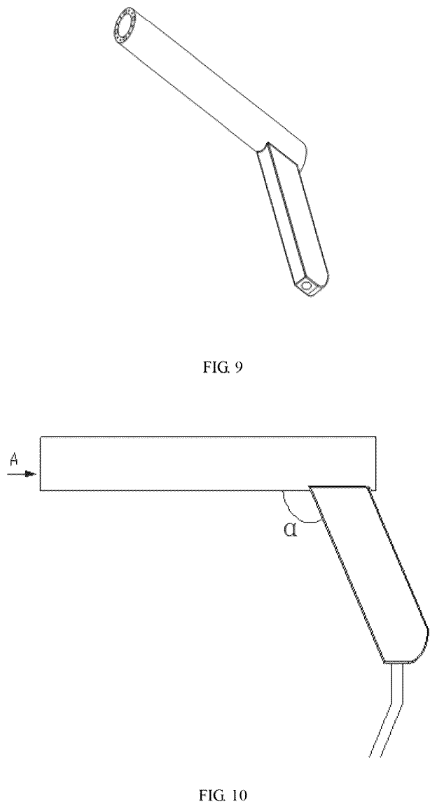

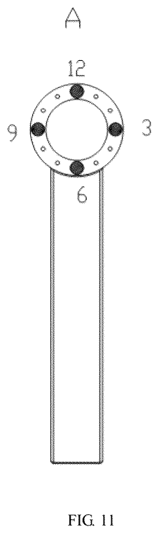

FIGS. 9, 10 and 11 exemplarily show the endoscope device 12 according to the aforementioned embodiments. As shown in FIGS. 9 and 10, the endoscope device 12 includes a tubular endoscope portion and a handle, below which a data cable extends and may be connected to a host. The handle and the tubular endoscope portion form an angle α, which may be any suitable angle, e.g. 120 degrees. FIG. 1l is a perspective view from the direction A in FIG. 10, showing that the outer tube of the tubular portion of the endoscope device 12 is divided into 12 equal parts, wherein the four image acquisition units are exemplarily located at 3 o'clock, 6 o'clock, 9 o'clock and 12 o'clock, respectively, and the plurality of illumination units are located at 1 o'clock, 2 o'clock, 4 o'clock, 5 o'clock, 7 o'clock, 8 o'clock, 10 o'clock and 11 o'clock, respectively.

The spacing between the plurality of image acquisition units and the spacing between the plurality of illumination units may also be unequal, for example, they may be set in a symmetrical manner or in other suitable manners.

The endoscopic illumination system and the camera system according to the aforementioned embodiments may ensure good surgical field during operation in the vascular lumen, avoiding the possibility that a single camera system cannot see the surgical field clearly because of a special angle and the obstruction of corresponding tissues.

According to a preferred embodiment of the present invention, the first balloon catheter (2F) and the second balloon catheter (4F) are fed in parallel alignment into the third balloon catheter (10F or 12F) and delivered to their respective blocking targets.

After the third balloon catheter is placed in place, it is convenient for the first and second balloon catheters to be placed in place. Further, catheter occupation between the third balloon 133 and the vascular wall in case of the transfemoral approach, caused by the parallel alignment, is eliminated, so that the third balloon 133 may be better directly attached to the vascular wall and the blocking is more accurately.

According to an embodiment of the present invention, a method for operating the aforementioned endovascular plaque excision system is provided, including: inserting the first balloon, the second balloon and the third balloon of the endovascular plaque excision system into the carotid artery, so that the first balloon is located at the opening of the external carotid artery of the affected side, the second balloon is located in the internal carotid artery of the affected side at the distal of the plaque, and the third balloon is located in the common carotid artery of the affected side at the proximal of the plaque; sequentially filling the second balloon, the first balloon, and the third balloon; and once blood flows back into the second balloon catheter, opening the blood shunting unit arranged in the second balloon.

Preferably, the method further includes: operating the intima stripping device after the first balloon, the second balloon and the third balloon are inflated. Preferably, the method further includes: operating the laser device to guide laser to the plaque in the carotid artery. Preferably, the method further includes: guiding irrigation fluid into the carotid artery.

According to another embodiment of the present invention, a method for excising a plaque in the common carotid artery is provided, including: puncturing left and right femoral arteries via the transfemoral approach, and placing artery sheaths respectively; placing the third balloon catheter into the common carotid artery of the affected side through the right femoral artery sheath, then placing the first balloon catheter into the external carotid artery of the affected side through the left femoral artery sheath, and then placing the second balloon catheter into the internal carotid artery of the affected side through the left femoral artery sheath, so that the first balloon is located at the opening of the external carotid artery of the affected side, the second balloon is located in the internal carotid artery of the affected side at the distal of the plaque, and the third balloon is located in the common carotid artery of the affected side at the proximal of the plaque; sequentially filling the second balloon, the first balloon, and the third balloon, opening the flank of a Y-type valve at the tail end of the second balloon catheter, and connecting the second balloon catheter with a communication tube of the left femoral artery sheath once blood is seen to flow back, so that the arterial blood in the left femoral artery sheath flows into the internal carotid artery of the affected side through the second balloon catheter; introducing the endoscope device into the common carotid artery through the third balloon catheter or introducing the endoscope device into the common carotid artery through the carotid sheath to reach the proximal of the plaque; placing the intima stripping device through the working lumen of the endoscope device and mechanically stripping the proximal of the plaque to separate it from the media, laser ablating the plaque tissue, and performing irrigating to make the debris flow out of the working lumen of the endoscope device.

In the aforementioned method, when the endoscope device is introduced into the common carotid artery through the carotid sheath, i.e. in case of the transcervical approach, the first balloon catheter, the second balloon catheter and the third balloon catheter may also be introduced in the following way: the third balloon catheter enters the right common carotid artery through the right femoral artery, and the first balloon catheter and the second balloon catheter enter the third balloon catheter in parallel alignment, and then enter the external carotid artery and the internal carotid artery respectively after exiting the third balloon catheter.

Preferably, the method for excising an arterial plaque further includes: continuously stripping the plaque distally, followed by laser ablating and irrigating until the plaque is completely excised under the endoscopic vision, and performing thorough irrigation to ensure that the surgical field is clean and free of debris. Preferably, the method for excising an arterial plaque further includes: after the excision is completed, keeping the tail end of the third balloon catheter open, first emptying the second balloon and then immediately filling it again, then emptying and withdrawing the first balloon, appropriately sucking the third balloon catheter, then emptying the third balloon, and finally emptying and withdrawing the second balloon. Preferably, the method for excising an arterial plaque further includes: performing angiography again through the third balloon catheter to confirm that the internal carotid artery and the intracranial artery are unobstructed.

It should be noted that the endovascular plaque excision system 10 according to the present invention is not limited to carotid plaque excision surgery, but may also be used in the field of peripheral blood vessels, such as aorta, mesenteric arteries, iliac arteries, atherosclerotic plaques in large-diameter blood vessels above the knee joint level of the lower limbs, and surgeries in other suitable fields.

The embodiments of the present invention are not limited to the aforementioned embodiments. Without departing from the spirit and scope of the present invention, those of ordinary skill in the art may make various changes and improvements to the form and details of the present invention, all of which are deemed to fall within the protection scope of the present invention.

1. An endovascular plaque excision system, comprising:
a balloon catheter system, adapted for being inserted into a blood vessel, comprising a guide catheter, a first balloon, a second balloon, and a third balloon, wherein the first balloon, the second balloon and the third balloon are arranged at a distal end, and adapted for being inflated to block blood flow in the blood vessel, and the second balloon comprises a blood shunting unit;
an endoscope device, adapted for being inserted into the blood vessel through the guide catheter for illumination and image acquisition, comprising an endoscope connecting tube, an illumination unit, and an image acquisition unit, wherein the illumination unit and the image acquisition unit are arranged at a distal end of the connecting tube; and
an intima stripping device, adapted for entering the blood vessel through the guide catheter to perform operation of stripping a plaque in the blood vessel, comprising an operation unit arranged at a proximal end, a stripping unit arranged at a distal end, and a stripping connecting tube which connects the operation unit with the stripping unit.

2. The endovascular plaque excision system according to claim 1, further comprising:
a laser device, comprising a laser generator and a laser transmission fiber, wherein the laser transmission fiber is adapted for entering the blood vessel through a fiber channel of the endoscope device, and transmitting the laser generated by the laser generator to a selected position for laser ablation of the plaque in the blood vessel.

3. The endovascular plaque excision system according to claim 1, further comprising:
a digital subtraction angiography machine, being capable of imaging the blood vessel, determining positions where the plaque is generated in the blood vessel, and identifying filling state of the balloons and positions where the balloons are located.

4. The endovascular plaque excision system according to claim 1, further comprising: a control device, controlling the balloons to be inflated and the laser generator to generate laser according to preset instructions.

5. The endovascular plaque excision system according to claim 1, it is adapted for excising a plaque in a carotid artery.

6. The endovascular plaque excision system according to claim 5, wherein the first balloon is adapted for being located at an opening of an external carotid artery of an affected side, the second balloon is adapted for being located in an internal carotid artery of the affected side at distal of the plaque, the third balloon is adapted for being located in a common carotid artery of the affected side at proximal of the plaque, and thereby, after the first balloon, the second balloon and the third balloon are inflated, a blood-free environment is formed in the blood vessel around the plaque.

7. The endovascular plaque excision system according to claim 6, wherein the blood shunting unit of the second balloon is in communication with arterial blood flow to convey the arterial blood flow to distal of the second balloon in an open state.

8. The endovascular plaque excision system according to claim 1, wherein the balloon catheter system comprises a first balloon catheter, a second balloon catheter, and a third balloon catheter; wherein the third balloon catheter is placed into the common carotid artery through a right femoral artery, and the first balloon catheter and the second balloon catheter are placed in parallel alignment into the external carotid artery and the internal carotid artery through a left femoral artery, respectively; and wherein the third balloon catheter comprises the guide catheter.

9.-10. (canceled)

11. The endovascular plaque excision system according to claim 1, wherein the endoscope device is provided with a catheter, which comprises a first lumen for accommodating the illumination unit and the image acquisition unit and a second lumen adapted for the passage of the laser transmission fiber and/or irrigation fluid, and a vascular wall protection device is provided at a distal end of the endoscope device; wherein there are a plurality of the image acquisition units, which are evenly and circumferentially distributed, and there are a plurality of the illumination units, which are arranged at equal intervals between each two adjacent image acquisition units, respectively; and wherein the endoscope device further comprises a third channel served as a working channel.

12.-13. (canceled)

14. An endovascular plaque excision system, comprising:
a balloon catheter system, adapted for being inserted into a blood vessel, comprising a guide catheter, a first balloon, a second balloon, and a third balloon, wherein the first balloon, the second balloon and the third balloon are arranged at a distal end, and are adapted for being inflated to block blood flow in the blood vessel, and the second balloon comprises a blood shunting unit;
an endoscope device, adapted for being inserted into the blood vessel through an artery sheath for illumination and image acquisition, comprising an endoscope connecting tube, an illumination unit, and an image acquisition unit, wherein the illumination unit and the image acquisition unit are arranged at a distal end of the connecting tube; and
an intima stripping device, adapted for entering the blood vessel through a working channel of the endoscope device to perform operation of stripping a plaque in the blood vessel, comprising an operation unit arranged at a proximal end, a stripping unit arranged at a distal end, and a stripping connecting tube which connects the operation unit with the stripping unit.

15. The endovascular plaque excision system according to claim 14, further comprising:
a laser device, comprising a laser generator and a laser transmission fiber, wherein the laser transmission fiber is adapted for entering the blood vessel through the working channel of the endoscope device, and transmitting the laser generated by the laser generator to a selected position for laser ablation of the plaque in the blood vessel.

16. The endovascular plaque excision system according to claim 14, further comprising:
a digital subtraction angiography machine, being capable of imaging the blood vessel, determining the position where the plaque is generated in the blood vessel, and identifying filling state of the balloons and the positions where the balloons are located.

17. The endovascular plaque excision system according to claim 14, further comprising: a control device, controlling the balloons to be inflated and the laser generator to generate laser according to preset instructions.

18. The endovascular plaque excision system according to claim 14, it is adapted for excising a plaque in a carotid artery.

19. The endovascular plaque excision system according to claim 18, wherein the first balloon is adapted for being located at an opening of an external carotid artery of an affected side, the second balloon is adapted for being located in an internal carotid artery of the affected side at distal of the plaque, the third balloon is adapted for being located in a common carotid artery of the affected side at proximal of the plaque, and thereby, after the first balloon, the second balloon and the third balloon are inflated, a blood-free environment is formed in the blood vessel around the plaque; and a carotid sheath is arranged in the common carotid artery at distal of a target blocked by the third balloon, so that the endoscope device is able to enter the common carotid artery and the internal carotid artery; and wherein the blood shunting unit of the second balloon is in communication with arterial blood flow to convey the arterial blood flow to distal of the second balloon in an open state.

20. (canceled)

21. The endovascular plaque excision system according to claim 14, wherein the balloon catheter system comprises a first balloon catheter, a second balloon catheter, and a third balloon catheter; wherein the third balloon catheter enters the right common carotid artery through a right femoral artery, and the first balloon catheter and the second balloon catheter enter the third balloon catheter in parallel alignment, and then enter the external carotid artery and the internal carotid artery respectively after exiting the third balloon catheter; and wherein the third balloon catheter comprises the guide catheter.

22.-23. (canceled)

24. The endovascular plaque excision system according to claim 14, wherein the endoscope device has a double-lumen design, comprising an inner lumen and an outer lumen which are coaxially arranged, wherein the inner lumen is optionally a working channel, through which a corresponding instrument, such as a plaque stripping instrument, a breaking instrument, or a grasping instrument, is able to be fed and the surgical field irrigation is able to be performed, and the outer lumen is adapted for serving as a layout channel for an endoscopic illumination system and a camera system; and wherein the endoscope device is provided with a plurality of the image acquisition units which are evenly and circumferentially distributed along the outer lumen of the endoscope device, and a plurality of the illumination units which are arranged at equal intervals between each two adjacent image acquisition units, respectively.

25.-29. (canceled)

30. A method for excising a plaque in a common carotid artery, comprising:
puncturing left and right femoral arteries via transfemoral approach, and placing artery sheaths thereinto respectively;
placing the third balloon catheter into the common carotid artery of an affected side through the right femoral artery sheath, then placing the first balloon catheter into an external carotid artery of the affected side through the left femoral artery sheath, and then placing the second balloon catheter into an internal carotid artery of the affected side through the left femoral artery sheath, so that the first balloon is located at an opening of the external carotid artery of the affected side, the second balloon is located in the internal carotid artery of the affected side at distal of the plaque, and the third balloon is located in the common carotid artery of the affected side at proximal of the plaque;
sequentially filling the second balloon, the first balloon, and the third balloon, opening a flank of a Y-type valve at a tail end of the second balloon catheter, and connecting the second balloon catheter with a communication tube of the left femoral artery sheath once blood is seen to flow back, so that arterial blood in the left femoral artery sheath flows into the internal carotid artery of the affected side through the second balloon catheter;
introducing an endoscope device into the common carotid artery through the third balloon catheter or introducing an endoscope device into the common carotid artery through a carotid sheath to reach the proximal of the plaque;
placing an intima stripping device through a working lumen of the endoscope device and mechanically stripping the proximal of the plaque to separate it from media, laser ablating the plaque tissue, and performing irrigation to make debris flow out of the working lumen of the endoscope device.

31. The method for excising a plaque in the common carotid artery according to claim 30, further comprising:
continuously stripping the plaque distally, followed by laser ablation and irrigation until the plaque is completely excised under the endoscopic vision, and performing thorough irrigation to ensure that the surgical field is clean and free of debris.

32. The method for excising a plaque in the common carotid artery according to claim 30, further comprising:
after the excision is completed, keeping a tail end of the third balloon catheter open, first emptying the second balloon and then immediately filling it again, then emptying and withdrawing the first balloon, appropriately sucking the third balloon catheter, then emptying the third balloon, and finally emptying and withdrawing the second balloon; and
performing angiography again through the third balloon catheter to confirm that the internal carotid artery and the intracranial artery are unobstructed.

33. (canceled)
